(19) United States
(12) Patent Application Publication
(10) Pub. No.: US 2024/0180495 A1
(43) Pub. Date: Jun. 6, 2024

(54) SYSTEMS AND METHODS FOR ASSESSING ECG LEAD SIGNAL QUALITY

(71) Applicant: InfoBionic, Inc., Chelmsford, MA (US)

(72) Inventors: Lev KORZINOV, San Diego, CA (US); Eric BAUMANN, San Diego, CA (US)

(73) Assignee: InfoBionic, Inc., Chelmsford, MA (US)

(21) Appl. No.: 18/488,219

(22) Filed: Oct. 17, 2023

Related U.S. Application Data
(60) Provisional application No. 63/380,405, filed on Oct. 21, 2022.

(51) Int. Cl. A61B 5/00; A61B 5/366

(52) U.S. Cl. CPC A61B 5/7221; A61B 5/366; A61B 5/7203

(57) ABSTRACT

A computer-implemented method for determining relative signal quality between electrocardiogram (ECG) leads may include: obtaining ECG data that includes a respective signal captured from a patient by each lead of a plurality of ECG leads; identifying a heartbeat in the ECG data; for each lead: extracting a portion of the respective signal corresponding to the identified heartbeat; identifying a heartbeat template most likely to correspond to the identified heartbeat; determining respective noise data for the extracted portion of the respective signal based on the identified heartbeat template; and determining a signal-to-noise ratio for the lead based on the respective noise data; and determining a lead from amongst the plurality of ECG leads having a highest signal-to-noise ratio.

### RELATED APPLICATIONS

This application is a non-provisional application of U.S. Provisional Application No. 63/380,405, filed on Oct. 21, 2022, and entitled “SYSTEMS AND METHODS FOR ASSESSING ECG LEAD SIGNAL QUALITY,” the disclosure of which is incorporated herein in its entirety.

### TECHNICAL FIELD

This application relates to electrocardiogram (“ECG”) monitoring and signal processing, and more particularly to determining relative signal quality between ECG leads and/or selecting one or more best or optimal ECG lead therefrom.

### BACKGROUND

An electrocardiogram (“ECG”) records the heart's electrical activity over time via electrodes placed on the skin. The electrical activity recorded by the electrodes may be converted into leads, e.g., signal representations of the heart's electrical activity. A lead may correspond to the signal response of an electrode, or may be derived from a combination of signals or leads. ECG results are generally interpreted by a medical professional who makes a judgment call as to the meaning of the results. An ECG may be used to detect various cardiac abnormalities or disturbances, e.g., an arrhythmia or a myocardial infarction.

Conventional techniques, including the foregoing, are generally at risk for inaccurate signals from the leads. Noise and other forms of inaccuracy can be introduced into ECG results in numerous ways. For example, the electrodes may produce various levels of noise depending manufacturing quality, electrode type, and placement on the skin. In another example, patient movement during an ECG may cause or change electrical activity recorded by the electrodes. Inconsistent or low signal quality may interfere with the accuracy and/or readability of ECG results, causing missed or inaccurate diagnoses.

This disclosure is directed to addressing one or more challenges, such as the above. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

According to certain aspects of the disclosure, methods and systems are disclosed for determining relative signal quality between ECG leads and/or selecting one or more best or optimal ECG lead therefrom.

In one aspect, a computer-implemented method for determining relative signal quality between electrocardiogram (ECG) leads may include: obtaining, via at least one processor, ECG data that includes a respective signal captured from a patient by each lead of a plurality of ECG leads; identifying, via the at least one processor, a heartbeat in the ECG data; for each lead: extracting, via the at least one processor, a portion of the respective signal corresponding to the identified heartbeat; identifying, via the at least one processor, a heartbeat template most likely to correspond to the identified heartbeat; determining, via the at least one processor, respective noise data for the extracted portion of the respective signal based on the identified heartbeat template; and determining, via the at least one processor, a signal-to-noise ratio for the lead based on the respective noise data; and determining, via the at least one processor, a lead from amongst the plurality of ECG leads having a highest signal-to-noise ratio.

In another aspect, a patient-specific heartbeat template library, may include at least one memory storing instructions; and at least one processor operatively connected to the at least one memory, and configured to execute the instructions to perform operations. The operations may include: receiving an extracted portion of a signal of electrocardiogram (ECG) data captured from a patient by an ECG lead, the extracted portion corresponding to a heartbeat; identifying a patient-specific heartbeat template stored in the at least one memory that is most likely to correspond to the identified heartbeat; and outputting the identified patient-specific heartbeat template.

In a further aspect, A system for determining relative signal quality between electrocardiogram (ECG) leads, may include at least one memory storing instructions; and at least one processor operatively connected to the at least one memory, and configured to execute the instructions to perform operations. The operations may include: obtaining ECG data that includes a respective signal captured from a patient by each lead of a plurality of ECG leads; identifying a heartbeat in the ECG data based on detection of a peak in the ECG data; for each lead: extracting a portion of the respective signal corresponding to the identified heartbeat; identifying a heartbeat template most likely to correspond to the identified heartbeat, by: accessing a patient-specific heartbeat template library; and comparing the extracted portion of the respective signal with any patient-specific heartbeat templates stored in the patient-specific heartbeat template library for the lead; and determining respective noise data for the extracted portion of the respective signal, by: removing a respective QRS complex from the extracted portion of the respective signal to form a respective signal remainder; obtaining a heartbeat template remainder in the form of the identified heartbeat template with a respective QRS complex removed; determining the respective noise data for the extracted portion of the respective signal based on a comparison between the respective signal remainder and the heartbeat template remainder; and determining a signal-to-noise ratio for the lead based on the respective noise data; and determining a lead from amongst the plurality of ECG leads having a highest signal-to-noise ratio.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts an exemplary system for assessing sensor device lead quality, according to one or more embodiments.

FIG. 2A depicts an exemplary method for assessing sensor device signal quality, according to one or more embodiments.

FIG. 2B depicts an exemplary schematic for extracting and analyzing various portions sensor device data that is usable with the method of FIG. 2A.

FIG. 3 depicts an exemplary method for generating templates, according to one or more embodiments.

FIG. 4 depicts a simplified functional block diagram of a computer, according to one or more embodiments.

### DETAILED DESCRIPTION

Generally, ECG lead signals may be impacted by different forms of noise. For example, even a slight motion of an electrode on a patient's skin, e.g., due to the patient's breathing, may interfere with an ECG reading. In another example, electrical activity generated in a patient's muscles, e.g., due to a patient moving their muscles, may interfere with an ECG reading. However, not all ECG leads may be equally affected by noise. As a result, it may be difficult or impossible to determine which lead provides a most-accurate indication of the heart's electrical activity at any given time. Accordingly, improvements in technology relating to determining lead-to-lead signal quality may be beneficial.

According to certain aspects of the disclosure, methods and systems are disclosed for determining relative lead quality. These methods and system may be usable to increase accuracy and/or sensitivity in equipment outputs and/or provide other benefits. As will be discussed in more detail below, in various embodiments, systems and methods are described that incorporate features such as template generation and/or noise analysis algorithms.

Reference to any particular activity is provided in this disclosure only for convenience and not intended to limit the disclosure. The disclosure may be understood with reference to the following description and the appended drawings, wherein like elements are referred to with the same reference numerals.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of ±10% of a stated or understood value.

It will also be understood that, although the terms first, second, third, etc. are, in some instances, used herein to describe various elements, these elements should not be limited by these terms. These terms are only used to distinguish one element from another. For example, a first contact could be termed a second contact, and, similarly, a second contact could be termed a first contact, without departing from the scope of the various described embodiments. The first contact and the second contact are both contacts, but they are not the same contact.

As used herein, the term “if” is, optionally, construed to mean “when” or “upon” or “in response to determining” or “in response to detecting,” depending on the context. Similarly, the phrase if it is determined” or “if [a stated condition or event] is detected” is, optionally, construed to mean “upon determining” or “in response to determining” or “upon detecting [the stated condition or event]” or “in response to detecting [the stated condition or event],” depending on the context.

Terms like “provider,” “medical provider,” or the like generally encompass an entity, person, or organization that may seek information, resolution of an issue, or engage in any other type of interaction with a patient, e.g., to provide medical care, medical intervention or advice, or the like. Terms like “patient,” “client,” or the like generally encompass any person or entity who is obtaining information, seeking resolution of an issue, or engaging in any other type of interaction with a medical provider, e.g., to receive such care, information, or the like. Terms like “user” generally encompass any person or entity that may obtain information, resolution of an issue, purchase of a product, or engage in any other type of interaction with a provider or patient, whereby the user may be the medical provider or the patient as the case may be. For example, an individual that operates an ECG on themselves may be both a patient and a user. In another example, a medical provider that operates an ECG on a patient may also be a user.

Terms such as “signal” or the like generally encompass any function that may convey information about a phenomenon. “Signals” may refer, for example, to any time varying voltage, current, or electromagnetic wave that carries information or an observable change in a quality, such as quantity, or may refer to the information itself. Terms such as “noise” or the like generally encompass extraneous, irrelevant, or relatively less meaningful data, or any data that is other than a signal intended to be observed. In the case of ECGs, “noise” may include, for example, unwanted signals that are merged with the ECG signal.

Terms such as “electrode” or the like generally encompass a conductive pad that is attached to the skin to record electrical activity. Terms such as “lead” or the like generally encompass a representation of the heart's electrical activity which may be calculated, for example, by analyzing data from several ECG electrodes. A “lead” may be graphically represented via an ECG signal, and may correspond to a signal from an electrode and/or may be derived. A “derived lead” generally encompasses a lead that is generated based on a programmatically and/or linearly weighted combination of leads.

In some instances, a medical provider may desire to use ECG results when providing care to a patient, e.g., for the purpose of making a diagnosis, determining a condition or status of the patient, or the like. ECG results may be obtained by using a plurality of ECG leads. For example, a medical provider may conduct an ECG and, in view of the patient's medical history, may believe the patient may be experiencing a ST-segment elevation myocardial infarction. However, the plurality of leads used to perform the ECG may provide varying ECG results relative to each other. Further, at any given time it may be difficult for the medical provider to determine which ECG lead may be producing the best quality signal, e.g., the lead that may be most representative of the electrical activity of the patient's heart. Conventionally, medical providers may use a judgment call to determine which lead may be producing the best signal. The judgment call may be based on comparing current ECG lead results to standardized ECG lead results based on a patient's history. For example, a medical provider may conduct an ECG on a patient and compare the patient's ECG lead results with standardized lead results. Standardized lead results are often outdated, are generally non-patient-specific, and may be significantly different from a current patient's ECG. The medical professional may visually or mentally compare the patient's lead results with the standardized lead results to determine which of the patient's leads is most similar to the standardized result. This comparison is inherently subjective, and may introduce inaccuracy into the medical provider's analysis or the patient's care if, for example, the medical provider relies mistakenly on a suboptimal lead. Thus, it may be beneficial to improve methods for determining lead signal quality, e.g., by measuring readings based on patient-specific templates.

In an exemplary use case, an analysis system may utilize templates to objectively determine which sensor device lead, e.g., which ECG lead, has the best signal quality. After the medical provider has set up the ECG, e.g., by adhering one or more electrodes to the patient's skin, the medical provider may operate the ECG. The ECG may collect data through the electrodes to generate a respective number of direct leads, e.g., two direct leads from two electrodes, and may calculate one or more derived leads, e.g., four derived leads. Calculation of the derived leads may be conducted by a computer, e.g., a computer housed within the ECG.

The ECG may use a peak detection algorithm to analyze the ECG leads to identify heartbeats and determine corresponding extraction windows. Based on the results of the peak detection algorithm, extraction windows may be generated around portions of the ECG signal corresponding to certain signals of interest, e.g., a 200 ms extraction window around a QRS complex (a combination of a Q wave, R wave, and S wave that represents ventricular depolarization) corresponding to or indicative of a heartbeat. An extraction algorithm may be used to extract the data within the extraction window (hereinafter referred to as “extracted portions”) from the ECG signal.

One or more lead-specific templates may be associated with the extracted portions. A template platform may be configured to generate and/or store one or more templates for each lead. A template may comprise a patient-specific ECG signal for a given lead, e.g., a 200 ms window including the QRS complex. The number of templates may depend on the number of leads. For example, the template platform may generate six templates for a six-lead ECG. The templates may be generated, e.g., by identifying a consistent heartbeat signal over one or more cardiac cycles, e.g., over ten cardiac cycles. Templates may be stored in a data storage system for future use. Patients may, in some instances, experience different heartbeats over time, e.g., due to an arrhythmia or other circumstances, and thus multiple templates corresponding to multiple different heartbeats may be generated or stored for each lead.

Templates may be compared to current ECG data to determine beat candidates, e.g., a particular template most likely to correspond to a particular heartbeat. The beat candidate may be determined by determining whether a similarity between the particular template and the particular heartbeat reaches at least a certain threshold value. If a beat candidate is not found, e.g., if the threshold is not met, a new template may be generated.

If a beat candidate is found, a removal algorithm may remove the QRS complex from the current ECG data and/or the best matching template. Removing the QRS complex may be used for and/or increase the accuracy of noise detection. The ECG data remaining once the QRS complex is removed (hereinafter referred to as “remaining portion”) may comprise the data surrounding the QRS complex, e.g., the P wave and the T wave, and, for at least the remaining portion of the current ECG data, may additionally include noise.

The amount of noise in the remaining portion, e.g., the amplitude of noise, may be determined by a noise determination algorithm, e.g., using a subtraction algorithm or a projection algorithm. For example, a QRS complex may be removed from the best matching template corresponding to the extracted portion of the current ECG data, and the resultant signal may be removed from the remaining portion (e.g., via subtraction or projection or the like), with the final result being an indication of noise in the ECG data.

Based on the output by the noise determination algorithm, the signal-to-noise ratio may be determined. The signal-to-noise ratio may be based on a comparison of an amplitude of the QRS complex signal of the extracted portion to an amplitude of noise determined by the noise determination algorithm, e.g., an amplitude of the final result discussed above. The signal-to-noise ratio for each lead may be compared to determine which lead has the highest signal-to-noise ratio. The highest signal-to-noise ratio may indicate the lead with the highest quality at any given time. The lead with the highest quality may vary over time. The process of determining the highest quality lead may be repeated periodically, e.g., every 10 seconds, such that the identification of the lead having the highest quality signal may be updated. The results from this process, i.e., which lead may have the highest quality at any given time, may be displayed by a graphical user interface (“GUI”). A GUI generally encompasses user interfaces that visually display information and may allow user interaction using electronic devices, graphical icons, and/or audio indicators.

While several of the examples above involve templates based on the QRS complex of an ECG, it should be understood that techniques according to this disclosure may be adapted to any suitable sensor device (e.g., electroencephalogram or ECG complex (e.g., ST segment, PR interval, etc.). It should also be understood that the examples above are illustrative only. The techniques and technologies of this disclosure may be adapted to any suitable activity. Presented below are various systems and methods of determining relative lead signal quality.

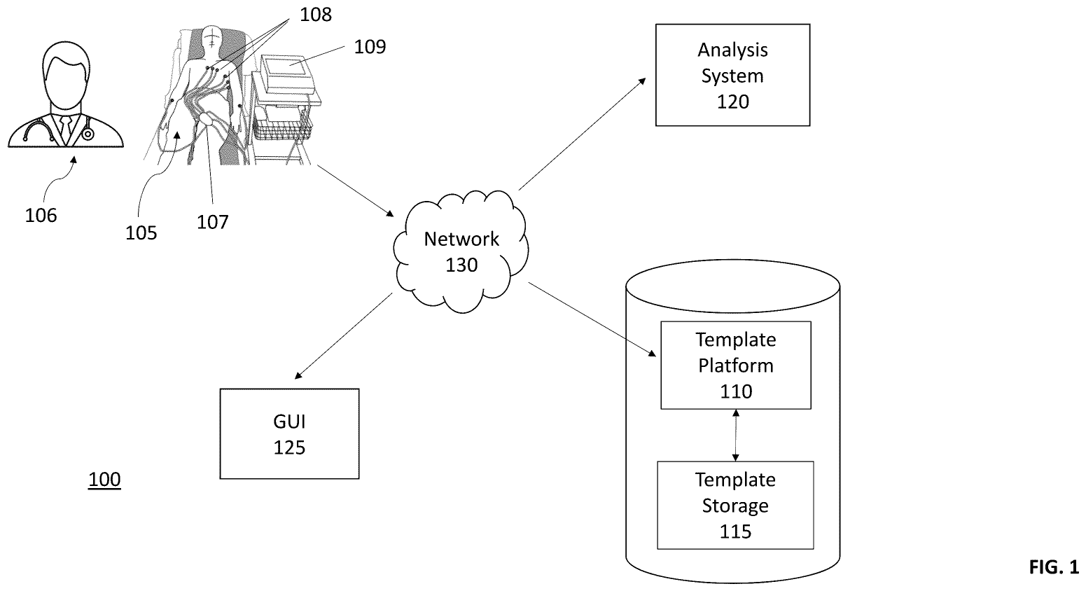

FIG. 1 depicts an exemplary system for assessing sensor device lead quality, according to one or more embodiments. Environment 100 of FIG. 1 may include a sensor device 107, a computer system 109, a template platform 110, a template storage 115, an analysis system 120, a GUI 125, and any of these may communicate over a network 130. Aspects of 100 may be operated by or with a patient 105 or a medical provider 106.

Sensor device 107 may include one or more sensors 108, e.g., one or more electrodes, that may be placed on the skin of patient 105 and configured to measure electrophysiology. Sensor device 107 may be in any form, including an ECG device having 2 leads, 3 leads, 5 leads, 8 leads, 12 leads, 15 leads, 16 leads, or any other suitable number of leads. The leads may be in any suitable form or combination of forms, such as direct or derived. For example, sensor device 107 may be an ECG with 2 direct leads and 4 derived leads. Sensor device 107 may be a provider-use ECG, for example a resting ECG, an exercise ECG, a signal-averaged ECG, an ambulatory monitoring ECG, an event-loop recording ECG, or any other type of ECG. Sensor device 107 could be an individual-use ECG, e.g., a portable ECG or any other type of ECG. Sensor device 107 may be configured to access and/or interact with other systems in environment 100. For example, sensor device 107 may incorporate, include, or be connected to the computer system, e.g., computer system 109, such as a desktop computer with or without a monitor, a mobile device, a tablet, etc. Sensor device 107 may also interact with template platform 110, template storage 115, analysis system 120, and/or GUI 125. In some embodiments, a data storage system, e.g., template storage 115, may be incorporated into sensor device 107.

Sensor device 107 may generate an output in the form of sensor device data, e.g., ECG data. Sensor device data may comprise data from one or more ECG leads, e.g., ECG data from six leads. Sensor device data may be communicated between one or more other components of the environment 100, e.g., between analysis system 120 and/or template platform 110.

Computer system 109 may include an internal storage system and/or may store data externally, e.g., in template storage 115. Computer system 109 may include any suitable input or output devices, e.g., a mouse, keyboard, buttons, a touch screen, a monitor, screen, or other form of display, speakers, lights, etc.

Template platform 110 may be configured to generate and/or store templates. Template generation algorithms may utilize the extracted portions to generate templates. Template platform 110 may output one or more templates per lead. In some embodiments, a patient may have multiple templates per lead, e.g., for a single lead, for each lead, or for any suitable number of leads. In an example where a patient has a cardiac arrhythmia, the patient may have templates associated with their normal cardiac rhythm and templates associated with their arrhythmic beat. By utilizing multiple templates, the analysis system may avoid inadvertently labeling an arrhythmic beat as noise.

Template platform 110 may output updated/new templates periodically and/or in response to failing to find a beat match, as described herein. For example, template platform 110 may output updated templates after a patient experiences pathological physiological changes (e.g., a heart attack), non-pathological physiological changes (e.g., athletic heart syndrome), and/or periodically at the direction of the user and/or in response to failing to detect a beat candidate.

Template storage 115 may include a server system, an electronic medical data system, computer-readable memory such as a hard drive, flash drive, disk, etc. In some embodiments, template storage 115 includes and/or interacts with an application programming interface for exchanging data to other systems, e.g., one or more of the other components of the environment 100. Template storage 115 may include and/or act as a repository or source for templates and/or for other forms of data, e.g., extracted portions. In some embodiments, template storage 115 may store a library of templates, e.g., a separate set of patient-specific templates for each of a plurality of patients. In some embodiments, template storage 115 may store one or more non-patient-specific templates.

Analysis system 120 may be configured to analyze ECG data to determine the highest quality lead. Analysis system 120 may output the signal-to-noise ratio for each lead and/or the determination of which lead has the highest quality. In some embodiments, analysis system 120 may include one or more peak determination algorithms, comparison algorithms, removal algorithms, noise determination algorithms, ratio algorithms, and/or other suitable algorithms. Peak determination algorithms may be configured to determine the peaks contained within sensor device data. These determinations may enable subsequently applied algorithms to determine signals of interest, e.g., heartbeats, within the sensor device data.

Comparison algorithms may be configured to compare sensor device data, lead data, or portions thereof, to one or more templates to determine a beat match. In some embodiments, a comparison algorithm may compare extracted portions of each lead to one or more templates associated with data from each lead in order to find a beat match. For example, a comparison algorithm may compare an extracted portion of Lead I to the one or more templates for Lead I to determine a beat match. If the comparison algorithm fails to find a beat match, template platform 110 may be directed to generate new templates. If the comparison algorithm finds a beat match, analysis system 120 may apply the removal algorithm to the ECG data and/or the best matching template. Further aspects of template matching and template generation are discussed below.

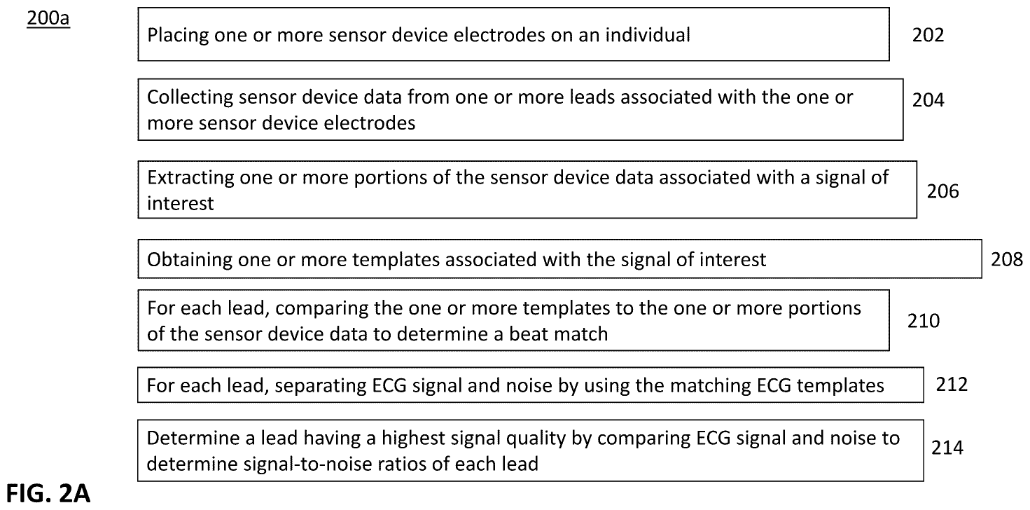

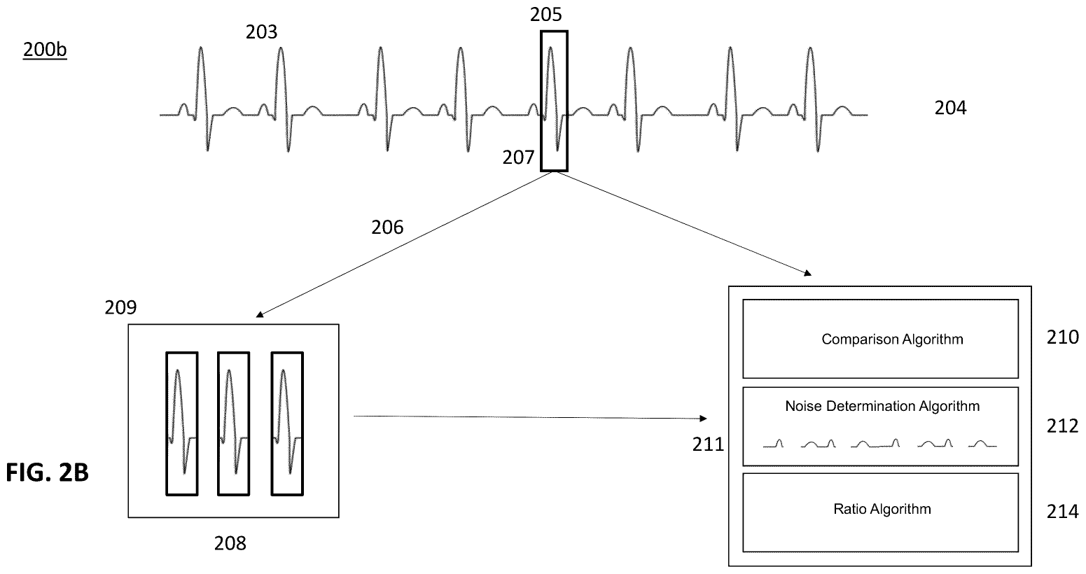

Removal algorithms may be configured to remove or extract one or more signals of interest, e.g., the QRS complex, from the ECG data and/or the best matching template. Removal algorithms may determine signals of interest based on the output of the peak determination algorithm. Removal algorithms may output a removed portion, e.g., the extracted QRS complex, and a remaining portion, e.g., the remaining ECG data, such as the P wave and T wave data, as described below in FIG. 2B.

Noise determination algorithms may determine the type and amplitude of noise within the remaining portion output from the removal algorithm. Noise determination algorithms may detect one or more types of noise, including but not limited to baseline wander, power line interference, electromyographic noise, electrode motion artifact noise, and/or any other form of noise.

Noise determination algorithms may determine the amplitude of noise in each lead by utilizing one or more algorithms, e.g., projection algorithms or subtraction algorithms. For example, the subtraction algorithm may subtract the template or a portion thereof from one or more portions of the ECG data, the output being the amount of noise within the ECG data. In another example, the projection algorithm may include projecting a best matching template onto the current ECG signal and excluding the ECG wave shape within of the wave shape of the template.

In one embodiment, QRS removal may be performed via a projection algorithm. In an example, a measured signal for a lead “n” may be denoted by S ri., a true (e.g., low- or no-noise) ECG signal for the lead “n” may be denoted by Eₙ, and a noise waveform may be denoted by Nₙ. The noise waveform Nₙ may be estimated, for example, via the following operations.

The measured signal Sₙ may be represented using the formula

Sₙ=Eₙ+Nₙ

A projection coefficient “c” may correspond to a template Tₙ^{m}, where “m” may denote the template number. Both vectors signal Sₙ and template Tₙ^{m} may be normalized to represent the projection coefficient “c” using the formula

c=Sₙ·Tₙ^{m}

Applying the Sₙ formula above to the projection coefficient “c” may be used to arrive at the formula

c=(Eₙ+Nₙ)·Tₙ^{m}=Eₙ·Tₙ^{m}+Nₙ·Tₙ^{m}

Since the noise waveform Nₙ may not be correlated with the QRS waveform, the second term of the above projection coefficient “c” formula, Nₙ·Tₙ^{m}, may have a value close to zero. Therefore, the projection coefficient “c” formula may be represented and/or approximated as

c=Eₙ·Tₙ^{m}

This formula may provide good accuracy, e.g., in predicting the projection coefficient “c.”

Since template Tₙ^{m} may have the same or similar shape as E n but a different amplitude, the true ECG signal Eₙ may be estimated using the formula

Eₙ=c·Tₙ^{m}

Based on the above derivations, the noise waveform Nₙ, may be calculated using the formula

Nₙ=Sₙ−Eₙ=Sₙ−c·Tₙ^{m}

Determining the noise waveform Nₙ, e.g., via the procedure above, may facilitate determination of one or more noise properties. For example, a standard deviation value may be determined, and may be used to estimate a signal-to-noise ratio. In this example, the measure for the signal-to-noise ratio may be a ratio of the standard deviation of Eₙ to the standard deviation of Nₙ. In other examples, the measure for the signal-to-noise ratio may be the ratio of the amplitude of Eₙ to the standard deviation of Nₙ.

Ratio algorithms may calculate the signal-to-noise ratios within each lead. Ratio algorithms may be configured to compare the QRS complex signal amplitude to the noise amplitude, the values derived from the removal algorithm and noise determination algorithm, respectively. The ratio algorithm may output the signal-to-noise ratio for each lead and/or which lead has the highest quality signal based on the ratio. This determination may be updated periodically, e.g., every 10 seconds.

GUI 125 may display data, e.g., which lead has the highest signal quality. The GUI may display one or more forms of data from one or more sources. For example, the GUI may display the ECG lead data, the signal-to-noise ratio for each lead, and/or the lead with the highest signal-to-noise ratio.

One or more of the components in FIG. 1 may communicate with each other and/or other systems, e.g., across network 130. In some embodiments, network 130 may connect one or more components of environment 100 via a wired connection, e.g., a USB connection between sensor device 107 and analysis system 120. In some embodiments, network 130 may connect one or more aspects of environment 100 via an electronic network connection, for example a wide area network (“WAN”), a local area network (“LAN”), personal area network (“PAN”), or the like. In some embodiments, the electronic network connection includes the Internet, and information and data provided between various systems occurs online. “Online” may mean connecting to or accessing source data or information from a location remote from other devices or networks coupled to the Internet. Alternatively, “online” may refer to connecting or accessing an electronic network (wired or wireless) via a mobile communications network or device. The Internet is a worldwide system of computer networks—a network of networks in which a party at one computer or other device connected to the network may obtain information from any other computer and communicate with parties of other computers or devices. The most widely used part of the Internet is the World Wide Web (often-abbreviated “WWW” or called “the Web”). A “website page,” a “portal,” or the like generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like. In any case, the connections within the environment 100 may be network, wired, any other suitable connection, or any combination thereof.

Although depicted as separate components in FIG. 1, it should be understood that a component or portion of a component in the environment 100 may, in some embodiments, be integrated with or incorporated into one or more other components. For example, analysis system 120 may be integrated in sensor device 107. In another example, analysis system 120 may further include a storage system, e.g., template storage 115, which may store sensor device data, templates, and/or other relevant data. In another example, template platform 110 and template storage 115 are distributed across one or more systems. In some embodiments, operations or aspects of one or more of the components discussed above may be distributed amongst one or more other components. Any suitable arrangement and/or integration of the various systems and devices of the environment 100 may be used.

FIG. 2A depicts an exemplary method 200a for assessing sensor device signal quality, e.g., ECG lead quality, according to one or more embodiments, and FIG. 2B depicts an exemplary schematic 200b for assessing sensor device signal quality that uses the method of FIG. 2A. At step 202 (FIG. 2A), a user (e.g., medical provider 106, patient 105, or any other third party user) may place one or more sensors 108 for sensor device 107 (e.g., ECG electrodes) on patient 105. At step 204, one or more sensors 108 for sensor device 107, e.g., ECG electrodes, may collect sensor device data 203 (FIG. 2B), e.g., via heart electrophysiology. For example, one or more peak detection algorithms may be used to identify peaks 205 in the ECG data signal and/or determine the structure of the ECG data signal. Such a determination may include identifying what portion of the ECG signal corresponds to a QRS complex by determining which peaks of the ECG correlate to the P wave, the R wave, and the T wave. In some embodiments, a predetermined period of time surrounding a detected peak is identified as a heartbeat. Based on the determination of the one or more peak detection algorithms, a signal of interest may be extracted from each lead. For example, a portion of the ECG data corresponding to a heartbeat may be extracted from each lead.

In some embodiments, sensor device 107 may be associated with a computer system 109. In some embodiments, computer system 109 may be configured to cause a display of the sensor device data, the templates, and/or other relevant data, as discussed in further detail below. The data on computer system 109 may be interfaced with via GUI 125. In some embodiments, sensor device data may be communicated between sensor device 107 and other aspects of environment 100, e.g., template platform 110 and/or analysis system 120.

At step 206, one or more portions of sensor device data associated with a signal of interest, e.g., the heartbeat, may be extracted from the ECG data, as an extracted portion 207. An extraction algorithm may be configured to conduct the extraction, as discussed herein. This extraction may be conducted by any aspect of environment 100, e.g., template platform 110 and/or analysis system 120.

At step 208, one or more templates 209 may be obtained by one or more aspects of environment 100, e.g., by analysis system 120. A template generation algorithm may generate and/or store templates based on the extracted portions. Templates may be generated by and/or obtained from template platform 110 and/or template storage 115, as discussed herein.

At step 210, for each lead, the one or more templates 209 may be compared to the one or more extracted portions 207 to determine a beat match. A beat match may be determined using a comparison algorithm to determine which template has the highest similarity to an extracted portion 207 to a given threshold. The process for determining the beat match between a template and extracted portion 207 may be repeated, e.g., for each heartbeat extracted from the ECG data and/or for each lead. In some embodiments, the beat match determination may be updated periodically, e.g., every 10 seconds. If a beat match is not determined, e.g., because the variation exceeds a threshold, one or more new templates may be generated based on the extracted portion 207 with no match and/or other similar extracted portions.

At step 212, the ECG signal may be separated from the noise for each lead using known ECG templates. In an example, using a removal algorithm, analysis system 120 may remove the QRS complex from the extracted portion and/or the best matching template. This process may result in a removed QRS complex and a remaining portion 211 (e.g., the signal that remains once the QRS complex is removed) for each lead. The removal algorithm and/or another algorithm may also determine the amplitude of the QRS complex.

A noise determination algorithm may be applied to the remaining portion 211 to determine characteristics of the noise, e.g., the types of noise found in the ECG signal, as well as the amplitude of noise. For example, after the removal algorithm may be applied to the corresponding best matching template, the remaining portion of the best matching template may be removed from the remaining portion 211 of the extracted portion 207, e.g., via subtraction or projection or the like. The result of the removal of the remaining portion of the template may be indicative of noise in the extracted portion 207.

At step 214, analysis system 120 may determine which lead may have the highest signal quality. For example, the analysis system may compare signal-to-noise ratios of each lead to determine which lead has the highest quality. As discussed in further detail below, the signal-to-noise ratio for a lead may be based on a comparison of the best matching template for a lead with the corresponding one or more portions of the sensor device data.

For example, the amplitude of the noise may be compared to the amplitude of the removed QRS complex from the extracted portion 207 using a ratio algorithm to determine the signal-to-noise ratio for each ECG lead. The signal-to-noise ratio for each lead may then be compared. For example, the signal-to-noise ratio for Lead I may be compared to the signal-to-noise ratio for Lead II and Lead III to determine which lead has the highest signal-to-noise ratio. The lead with the highest signal-to-noise ratio may have the highest quality.

Determination of the highest signal-to-noise ratio may be repeated. In some instances, the lead that has the highest signal quality may change at any given time. Thus, in some embodiments, at least a portion of the method above may be repeated periodically, e.g., every 10 seconds. Portions of the method may, in various embodiments, be repeated in response to a user instruction, in response to detection of a variance or change in signal data from a lead or leads, or for any other suitable reason. In this manner, at any given time, the lead having the highest quality signal may be identified.

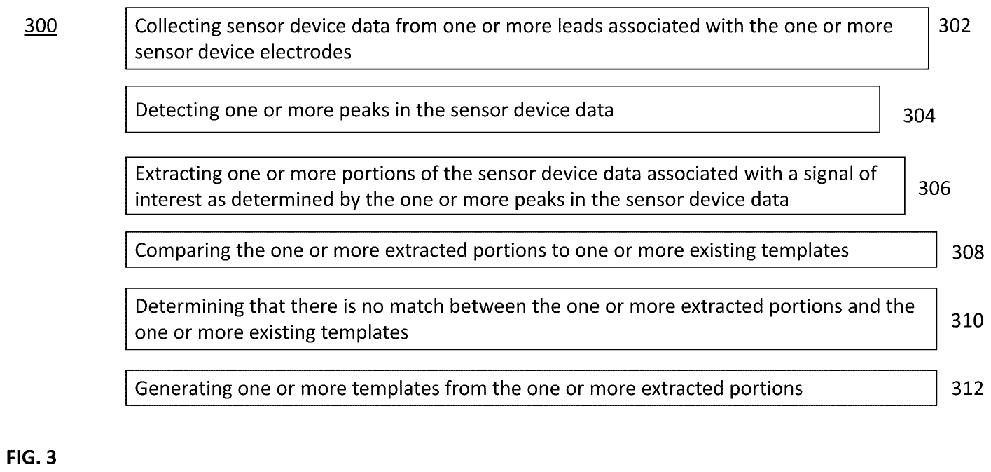

FIG. 3 depicts an exemplary method for generating templates, according to one or more embodiments. At step 302, sensor device data may be collected from one or more leads associated with one or more sensor device electrodes. For example, 10, 30, 60 seconds or the like of data, a predetermined number of heartbeats, or the like may be acquired. At step 304, one or more peaks may be detected in the sensor device data. As discussed herein, the peaks may be detected by any aspect of environment 100, e.g., computer system 109, analysis system 120, etc. Peak detection may enable system to determine signals of interest, e.g., heartbeats. For example, the system may be able to determine what portion of the ECG signal corresponds to the QRS complex by determining which peaks of the ECG correlate to the P wave, the R wave, and the T wave. In another example, a predetermined period of time, e.g., 200 ms may be associated with a heartbeat.

At step 306, one or more portions of the sensor device data, e.g., heartbeats, may be extracted, as discussed herein. For example, an extraction algorithm may be configured to extract one or more heartbeats from the ECG data as extracted portions. At step 308, one or more of the extracted portions may be compared to existing templates in a template storage 115. However, it should be understood that in some instances there may be no existing templates in the template storage 115. At step 310, the template platform 110 may determine that there is no match between the one or more extracted portions and an existing template.

At step 312, in response to determining that there is no matching template, one or more templates may be generated from the one or more extracted portions. One or more template generation algorithms may be configured to generate templates using, for example, a clustering algorithm. A clustering algorithm may cluster two or more extracted portions based on their relatedness and/or similarity. The clustering algorithm may also cluster extracted portions based on the dissimilarity of the extracted portions to other established clusters. Templates may be generated by averaging each cluster to determine cluster-specific templates. For example, a template may be generated based on averaging at least 10 clustered extracted portions for a particular lead.

In an example, a clustering algorithm may be implemented by setting a maximum distance between a current QRS waveform and a known cluster. In this example, the current QRS waveform may be compared to all templates, and if the closest template is different more than a maximum tolerance threshold, then a new template and/or cluster may be created from the current waveform.

More specifically, given the waveform Eₙ, the algorithm may calculate the distance D^{m} for each template Tₙ^{m} using the formula

D^{m}=|Eₙ−Tₙ^{m}|

The lowest distance D^{m} may have the index “m” of the closest template. If this D^{m} is below the tolerance threshold, then the current QRS waveform may be considered a part of and/or added to a cluster associated with the closest template, and/or the corresponding template may be identified as representative of the current QRS waveform. If D^{m} is above the tolerance threshold, then a new template and/or new cluster may be created.

Any other suitable clustering algorithm or algorithms may be used, including, but not limited to: K-means clustering, Density-Based Spatial Clustering of Applications with Noise (DBSCAN) algorithm, Gaussian mixture model, mean shift algorithm, affinity propagation clustering, spectral clustering, etc.

The one or more templates may consist of data relating to an extracted complex axis, an extracted complex width, a percent the complex is premature, any other suitable parameter, and/or any suitable combination thereof. For example, a template may comprise a QRS complex axis, a QRS complex width, and the percent the QRS complex is premature.

The templates may be updated and/or changed. In some embodiments, templates may be updated in response to the failure to determine a beat match. For example, a patient may require updated templates after a myocardial infarction. The damage caused by a myocardial infarction may cause permanent physiological changes to a patient's heart, changes which may alter the ECG signal. As such, the patient may have pre-myocardial infarction templates, which may no longer match the patient's post-myocardial infarction templates. In some embodiments, the templates may be updated by request of the user, e.g., the medical professional, and/or in response to detecting a threshold change in a patient's ECG, e.g., the template platform detects a threshold change in the ECG signal after failing to determine a beat match.

In some embodiments, templates may be updated periodically. A patient's heartbeat may change over time due to pathological changes (e.g., hypertension), non-pathological changes (e.g., athletic heart syndrome), and/or any other causes of cardiac function changes. The templates may be updated periodically to account for these changes. Templates may be updated and/or changed manually, e.g., in response to a request from a medical professional, or automatically, e.g., every year.

In an example, updating one or more templates may include re-clustering extracted portions to take into account newly added extracted portions, remove extracted portions over a predetermined age or over a threshold dissimilarity from newly acquired extracted portions, re-averaging clusters, etc.

The one or more templates may be generated and/or stored by template platform 110. The one of more templates may be associated with particular leads. For example, a template generated from Lead I may be associated with comparisons to current Lead I data, a template generated from Lead II may be associated with comparisons to current Lead II data, and so on. Template storage 115 may store the one or more templates.

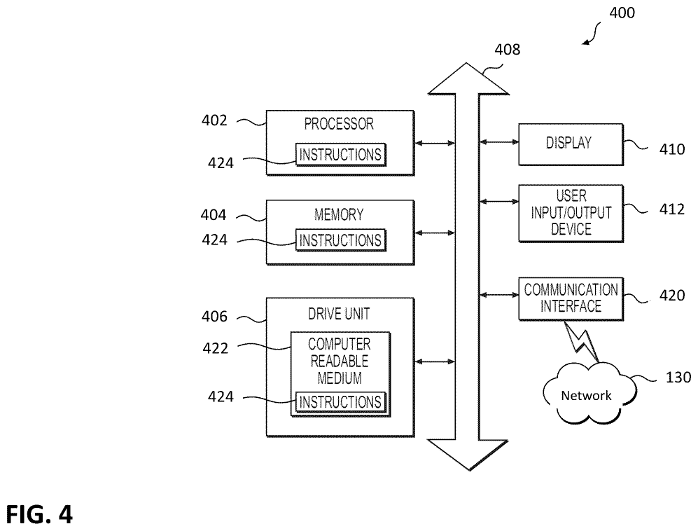

FIG. 4 depicts a simplified functional block diagram of a computer 400 that may be configured as a device for executing the methods of FIGS. 2A, 2B, and 3 according to exemplary embodiments of the present disclosure. One or more of a processor 402, a memory 404, a drive unit 406, an internal communication bus 408, a display 410, a under input/output ports 412, a communication interface 420, a computer readable medium 422, instructions 424, and a network 130 may communicate by any suitable means. For example, computer 400 may be configured as sensor device 107, template platform 110, template storage 115, analysis system 120, and/or another system according to exemplary embodiments of this disclosure. In various embodiments, any of the systems herein may be a computer 400 including, for example, data communication interface 420 for packet data communication. Computer 400 also may include a central processing unit (“CPU”) 402, in the form of one or more processors, for executing program instructions. Computer 400 may include internal communication bus 408, and storage unit 406 (such as Read-Only Memory (“ROM”), Hard Disk Drive (“HDD”), Solid-State Drive (“SSD”), etc.) that may store data on computer readable medium 422, although computer 400 may receive programming and data via network communications. Computer 400 may also have memory 404 (such as Random-Access Memory (“RAM”)) storing instructions 424 for executing techniques presented herein, although instructions 424 may be stored temporarily or permanently within other modules of computer 400 (e.g., processor 402 and/or computer readable medium 422). Computer 400 also may include input and output ports 412 and/or display 410 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While the disclosed methods, devices, and systems are described with exemplary reference to transmitting data, it should be appreciated that the disclosed embodiments may be applicable to any environment, such as a desktop or laptop computer, an automobile entertainment system, a home entertainment system, medical equipment, etc. Also, the disclosed embodiments may be applicable to any type of Internet protocol.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments may be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

We claim:

1. A computer-implemented method for determining relative signal quality between electrocardiogram (ECG) leads, comprising:
obtaining, via at least one processor, ECG data that includes a respective signal captured from a patient by each lead of a plurality of ECG leads;
identifying, via the at least one processor, a heartbeat in the ECG data;
for each lead:
extracting, via the at least one processor, a portion of the respective signal corresponding to the identified heartbeat;
identifying, via the at least one processor, a heartbeat template most likely to correspond to the identified heartbeat;
determining, via the at least one processor, respective noise data for the extracted portion of the respective signal based on the identified heartbeat template; and
determining, via the at least one processor, a signal-to-noise ratio for the lead based on the respective noise data; and
determining, via the at least one processor, a lead from amongst the plurality of ECG leads having a highest signal-to-noise ratio.

2. The computer-implemented method of claim 1, wherein the heartbeat in the ECG data is identified based on detection of a peak in the ECG data.

3. The computer-implemented method of claim 2, wherein the portion of the respective signal corresponding to the identified heartbeat is determined based on detection of a QRS complex associated with the detected peak.

4. The computer-implemented method of claim 1, wherein identifying the heartbeat template most likely to correspond to the identified heartbeat includes:
accessing a patient-specific heartbeat template library; and
comparing the extracted portion of the respective signal with any patient-specific heartbeat templates stored in the patient-specific heartbeat template library for the lead.

5. The computer-implemented method of claim 4, wherein:
the patient-specific heartbeat template library is configured to:
collect, for each lead, extracted portions of the respective signal of the lead; and
separate the collected extracted portions into one or more clusters; and
each of the clusters corresponds to a respective patient-specific heartbeat template.

6. The computer-implemented method of claim 5, wherein:
comparing the extracted portion of the respective signal with any patient-specific heartbeat templates stored in the patient-specific heartbeat template library for the lead includes performing a clustering operation on the extracted portion of the respective signal to determine a cluster from amongst the one or more clusters that is a closest match to the extracted portion; and
the respective patient-specific heartbeat template corresponding to the cluster that is the closest match is identified as the heartbeat template most likely to correspond to the identified heartbeat.

7. The computer-implemented method of claim 4, wherein identifying the heartbeat template most likely to correspond to the identified heartbeat further includes:
determining that no patient-specific heartbeat template stored in the patient-specific heartbeat template library for the lead is likely to correspond to the identified heartbeat;
in response to determining no patient-specific heartbeat template is likely to correspond to the identified heartbeat, generating a new patient-specific heartbeat template based on the extracted portion of the respective signal; and
identifying the new patient-specific heartbeat template as the heartbeat template most likely to correspond to the identified heartbeat.

8. The computer-implemented method of claim 1, wherein determining the respective noise data for the extracted portion of the respective signal based on the identified template includes:
removing a respective QRS complex from the extracted portion of the respective signal to form a respective signal remainder;
obtaining a heartbeat template remainder in the form of the identified heartbeat template with a respective QRS complex removed; and
determining the respective noise data for the extracted portion of the respective signal based on a comparison between the respective signal remainder and the heartbeat template remainder.

9. The computer-implemented method of claim 8, wherein determining the signal-to-noise ratio for the lead based on the respective noise data includes comparing the respective noise data to the respective QRS complex of the extracted portion of the respective signal.

10. The computer-implemented method of claim 1, wherein the obtaining step, the identifying step, and the step of determining the lead having the highest signal-to-noise ratio are periodically repeated.

11. The computer-implemented method of claim 10, further comprising:
causing a graphical-user-interface of a display to output an indication, for each instance in which the obtaining step, the identifying step, and the step of determining the lead having the highest signal-to-noise ratio are periodically repeated, which lead of the plurality of ECG leads has the highest signal-to-noise ratio for that instance.

12. A patient-specific heartbeat template library, comprising:
at least one memory storing instructions; and
at least one processor operatively connected to the at least one memory, and configured to execute the instructions to perform operations, including:
receiving an extracted portion of a signal of electrocardiogram (ECG) data captured from a patient by an ECG lead, the extracted portion corresponding to a heartbeat;
identifying a patient-specific heartbeat template stored in the at least one memory that is most likely to correspond to the identified heartbeat; and
outputting the identified patient-specific heartbeat template.

13. The patient-specific heartbeat template library of claim 12, wherein the operations further include, for each of one or more ECG leads:
collecting extracted portions of respective signals of ECG data captured from the patient;
separating the collected extracted portions into one or more clusters; and
generating a respective patient-specific heartbeat template based on the extracted portions included in each cluster.

14. The patient-specific heartbeat template library of claim 13, wherein:
identifying the patient-specific heartbeat template stored in the at least one memory that is most likely to correspond to the heartbeat includes determining a cluster from amongst the one or more clusters that is a closest match to the extracted portion.

15. The patient-specific heartbeat template library of claim 13, wherein identifying the patient-specific heartbeat template stored in the at least one memory that is most likely to correspond to the heartbeat includes:
determining that no patient-specific heartbeat template stored in the at least one memory for the ECG lead is likely to correspond to the heartbeat;
in response to determining no patient-specific heartbeat template is likely to correspond to the heartbeat, generating a new patient-specific heartbeat template based on the extracted portion of the signal; and
identifying the new patient-specific heartbeat template as the heartbeat template most likely to correspond to the heartbeat.

16. The patient-specific heartbeat template library of claim 15, wherein generating the new patient-specific heartbeat template includes:
adding the extracted portion of the signal to the collected extracted portions;
repeating the separating of the collected extracted portions into one or more clusters; and
generating the new patient-specific heartbeat template based on the extracted portions included in a cluster including the extracted portion of the signal.

17. The patient-specific heartbeat template library of claim 16, wherein generating the new patient-specific heartbeat template further includes obtaining a plurality of further extracted portions of the signal and adding the plurality of further extracted portions to the collected extracted portions.

18. The patient-specific heartbeat template library of claim 16, wherein generating the new patient-specific heartbeat template further includes removing at least one extracted portion from the collected extracted portions based on one or more of an age of the at least one extracted portion or a dissimilarity between the at least one extracted portion to be removed and the received extracted portion of the signal.

19. A system for determining relative signal quality between electrocardiogram (ECG) leads, comprising:
at least one memory storing instructions; and
at least one processor operatively connected to the at least one memory, and configured to execute the instructions to perform operations, including:
obtaining ECG data that includes a respective signal captured from a patient by each lead of a plurality of ECG leads;
identifying a heartbeat in the ECG data based on detection of a peak in the ECG data;
for each lead:
extracting a portion of the respective signal corresponding to the identified heartbeat;
identifying a heartbeat template most likely to correspond to the identified heartbeat, by:
accessing a patient-specific heartbeat template library; and
comparing the extracted portion of the respective signal with any patient-specific heartbeat templates stored in the patient-specific heartbeat template library for the lead; and
determining respective noise data for the extracted portion of the respective signal, by:
removing a respective QRS complex from the extracted portion of the respective signal to form a respective signal remainder;
obtaining a heartbeat template remainder in the form of the identified heartbeat template with a respective QRS complex removed;
determining the respective noise data for the extracted portion of the respective signal based on a comparison between the respective signal remainder and the heartbeat template remainder; and
determining a signal-to-noise ratio for the lead based on the respective noise data; and
determining a lead from amongst the plurality of ECG leads having a highest signal-to-noise ratio.

20. The system of claim 19, wherein determining the signal-to-noise ratio for the lead based on the respective noise data includes comparing the respective noise data to the respective QRS complex of the extracted portion of the respective signal.
